(19) United States
(12) Patent Application Publication
(10) Pub. No.: US 2004/0131155 A1
(43) Pub. Date: Jul. 8, 2004

(54) X-ray diagnostic system with a CCD camera

(76) Inventor: Anton Nekovar, Neunkirchen (DE)

Correspondence Address: HARNESS, DICKEY & PIERCE, P.L.C., P.O.BOX 8910, RESTON, VA, 20195, US

(21) Appl. No.: 10/705,908

(22) Filed: Nov. 13, 2003

(30) Foreign Application Priority Data
Nov. 13, 2002 (DE) 10253076.9

(51) Int. Cl. H05G 1/64

(52) U.S. Cl. 378/98.8

(57) ABSTRACT

An X-ray diagnostic system includes a CCD camera, a device for generating external trigger pulses and a system control. The system control is formed in such a way that, in the absence of X-radiation, a readout of the CCD camera without a useful signal takes place at regular time intervals. To reduce the dark signal component in the useful signal of the CCD camera, the system control is formed in such a way that, when an external trigger pulse occurs at a point in time at which no readout of the CCD camera is taking place, firstly a readout without a useful signal is triggered and then an exposure of the CCD camera takes place. Further, when an external trigger pulse occurs at a point in time at which a readout of the CCD camera is taking place, a readout without a useful signal is suppressed before an exposure of the CCD camera.

The present application hereby claims priority under 35 U.S.C. §119 on German patent application number DE 102 53 076.9 filed Nov. 13, 2002, the entire contents of which are hereby incorporated herein by reference.

### FIELD OF THE INVENTION

The invention generally relates to an X-ray diagnostic system with a CCD camera. More preferably, it relates to a device for generating external trigger pulses and a system control, which is formed in such a way that, in the absence of X-radiation, a readout of the CCD camera without a useful signal takes place at regular time intervals.

### BACKGROUND OF THE INVENTION

DE 44 24 905 C1 discloses a X-ray diagnostic system with a CCD image converter, in which the useful charge of the CCD image converter is scanned twice. Further, in the event of overadjustment, the pixel signal of the first scan is replaced by a correspondingly adapted signal of the second scan.

In the case of such X-ray diagnostic systems, however, the problem arises that, depending on the time period between the last scan and the current scan, a dark signal is accumulated on the CCD image converter. This has the result that different dark signal conditions are obtained in the case of the first picture taken than in the case of the ones which follow, which follow more or less regularly at fixed intervals.

It is already known in the absence of X-radiation to read out the CCD camera without a useful signal at regular time intervals in order that the dark signal component is reduced. However, here, too, there are irregular intervals between the last readout without a useful signal and the first exposure and between the readouts with a useful signal.

### SUMMARY OF THE INVENTION

An embodiment of the invention is based on an object of forming an X-ray diagnostic system in such a way that the dark signal component in the pictures taken is greatly reduced and is virtually the same for all the cyclically following pictures.

An embodiment of the invention achieves the effect that, with a relatively great interval between a picture-taking pulse and the last readout pulse of the CCD camera, a virtually uniform interval is achieved.

It has proven to be advantageous if, when an external trigger pulse occurs at a point in time at which a readout of the CCD camera is taking place, the X-ray diagnostic system is immediately triggered for the emission of X-radiation and the useful signal is subsequently read out.

In an advantageous way, when an external trigger pulse occurs at a point in time at which no readout of the CCD camera is taking place, firstly a read out without a useful signal can be carried out and then the X-ray diagnostic system triggered for the emission of X-radiation.

According to an embodiment of the invention, the device for generating external trigger pulses may be an ECG electrode with an associated control or a phase-angle sensor.

### BRIEF DESCRIPTION OF THE DRAWINGS

The present invention will become more fully understood from the detailed description of preferred embodiments given hereinbelow and the accompanying drawings, which are given by way of illustration only and thus are not limitative of the present invention, and wherein:

FIG. 1 shows a known X-ray diagnostic system with an X-ray detector,

FIGS. 2 to 5 show curves of the clock and control signals of a known X-ray diagnostic system, of which

FIG. 2 shows triggering of the CCD camera 8,

FIG. 3 shows a readout signal of the CCD camera 8,

FIG. 4 shows an external trigger,

FIG. 5 shows X-radiation,

FIGS. 6 to 14 show curves of the clock and control signals of an X-ray diagnostic system according to an embodiment of the invention, of which

FIG. 6 shows reset triggering of the CCD camera 8,

FIG. 7 shows dark readout of the CCD camera 8,

FIG. 8 shows an external trigger of the X-ray diagnostic system,

FIG. 9 shows X-radiation of the X-ray diagnostic system,

FIG. 10 shows readout pulses of the CCD camera 8,

FIG. 11 shows readout signals of the CCD camera 8,

FIG. 12 shows an external trigger of the X-ray diagnostic system,

FIG. 13 shows the camera trigger of the CCD camera 8 and

FIG. 14 shows the control signal for the X-radiation.

### DETAILED DESCRIPTION OF THE PREFERRED EMBODIMENTS

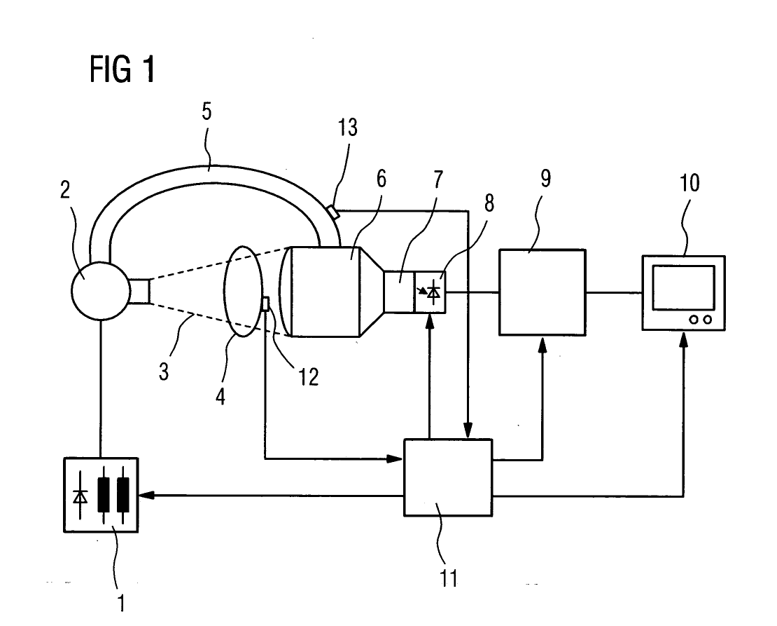

Represented in FIG. 1 is an X-ray tube 2 which is operated by a high-voltage generator 1 and emits an X-ray beam 3, which passes through a patient 4. The X-ray tube 2 is mechanically coupled to an X-ray image amplifier 6 by means of a C arch. Instead of the rigid mechanical coupling by the C arch 5, however, electronic coupling may also be provided if the X-ray tube 2 and the X-ray image amplifier 6 are, for example, arranged on freely positionable telescopic arms.

The fluorescent output screen of the X-ray image amplifier 6 is coupled via an optical system 7 to a CCD camera 8, the output signal of which is fed to an image system 9 connected for display purposes to a monitor 10. The components are controlled in their time sequences by a system control and communication 11. Fastened to the patient 4 is an ECG electrode 12, the output signal of which is connected to the control of the high-voltage generator (as still to be explained further). If this X-ray diagnostic system can be used to produce radiographs for rotation angiography, for example, a phase-angle sensor 13 which is likewise connected to the system control 11 can be attached to the C arch 5.

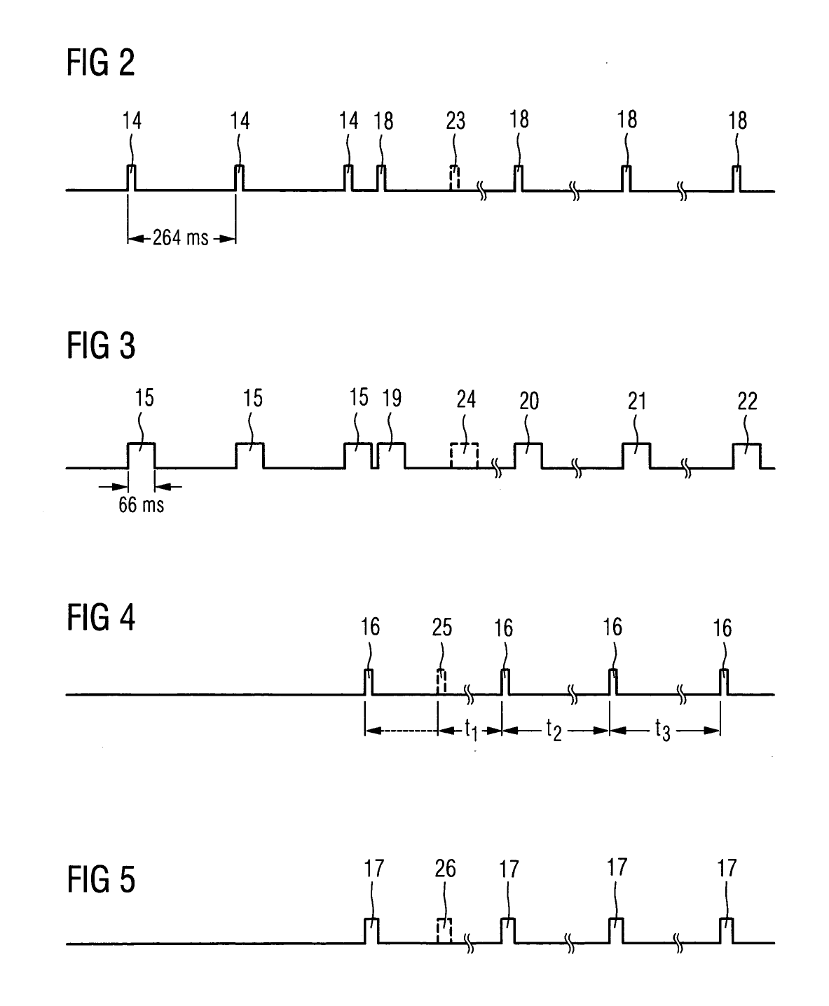

The clock and control signals of an X-ray diagnostic system according to the prior art are explained in more detail on the basis of FIGS. 2 to 5.

Shown in FIG. 2 is the triggering of the CCD camera 8. For example, a reset pulse 14, which brings about an automatic reset, is generated every 264 ms. This reset is intended to prevent an excessively high dark signal from accumulating on the CCD image converter. These reset pulses 14 result in a readout of the CCD image converter, shown in FIG. 3, which is represented as a dark readout 15. In our example, this readout time of the camera is 66 ms.

Shown in FIG. 4 is the external trigger, which is generated for example from the ECG captured by the ECG electrode 12 of a person being examined or on the basis of an angle triggering controlled by the phase-angle sensor 13. On the basis of this trigger pulse 16, the radiation pulses 17 of the X-radiation represented in FIG. 5 are generated, at the end of which, controlled by an exposure control which is not shown, a readout pulse 18 is set with the camera trigger, whereby the CCD image converter is read out, as shown by the readout signals 19 to 22 in FIG. 3.

In the case represented, the interval of the external trigger pulse 16 from the automatic reset pulse 14 is very much smaller than 264 ms, so that the dark signal component makes up a very small proportion of the first readout signal 19.

If the external trigger pulse 16 then occurs at a later point in time within the reset interval of 264 ms, the dark signal increases and, in the case in which averaging takes place over almost 260 ms and which is represented by the dashed pulses 23 to 26, is very high and disruptive. The pulses 23 to 26 occur instead of the pulses 16 to 19.

The further external trigger pulses 16 control the radiation and after that the readout of the CCD image converter in a known way. These external trigger pulses 16 may occur at irregular times, since in the first example they are controlled by the ECG and in the second example, when there is a movement of the image generating system, they depend on the speed during acceleration or braking. As a result, different times t₁ to t₃ are also obtained between the individual camera trigger pulses, so that the dark signal of the corresponding readouts can also vary correspondingly.

The image converter of the CCD camera 8 is an IT image converter (interline transfer), in which the charge accumulated in the light-sensitive region is transferred by a trigger pulse (14, 18 or 23) within the shortest time (for example ≦300 μs) into a memory area which is shielded from the incidence of light; after that, the actual exposure of the light-sensitive region of the CCD image converter can then immediately take place. From this memory area, the actual readout of the charge corresponding to the exposure from the shielded region also takes place immediately and is then fed to the image system 9 as a video signal. This readout operation lasts 66 ms.

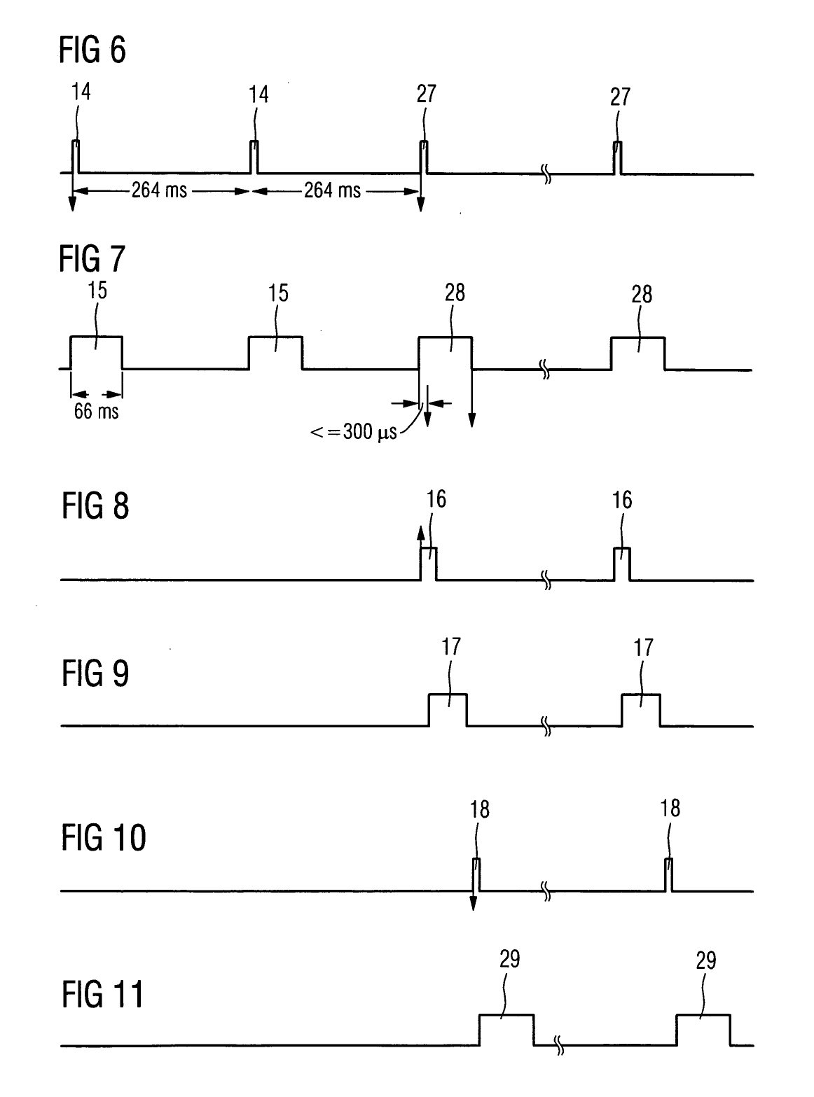

The operating principle of the X-ray diagnostic system according to the invention is now explained in more detail on the basis of the clock and control signals represented in FIGS. 6 to 11. Shown in FIG. 6 is the reset triggering of the CCD camera 8 with the reset pulses 14. On the basis of these reset pulses 14, recurring every 264 ms, a dark readout 15 takes place, which is represented in FIG. 7. If in the case of the external trigger in FIG. 8 there occurs a trigger pulse 16, which may be triggered by the ECG electrode 12 and/or the phase-angle sensor 13, this brings about a reset pulse 27, which results in a dark readout 28.

Triggered with an offset time span of less than or equal to 300 μs is a radiation pulse 17 (represented in FIG. 9), the length of which is dependent on the thickness of the subject under examination (is correspondingly corrected). Generated on completion of either the dark readout 28 or the radiation pulse 17 is a readout pulse 18 (represented in FIG. 10), which brings about a readout of the image, as shown by the readout signals 29 represented in FIG. 11.

However, this results in a dark readout 28 taking place immediately before a readout signal 29. Thus, the dark signal component contained in the useful signal is always the same and very small, irrespective of the time spans t₁ to t₃.

The IT image converter of the CCD camera 8 is consequently activated in such a way that, with the reset pulse 27, the dark charge accumulated in the light-sensitive region is transferred within a time of for example 300 ms into the memory area shielded from the incidence of light. This is followed by the exposure of the light-sensitive region of the CCD image converter during the radiation pulses 17. At the same time, the dark signal is read out from the memory area. After this dark readout 28, the transfer of the charge image produced during the actual exposure into the memory area takes place with the readout pulse 18. From this memory area, the actual readout of the charge from the shielded region corresponding to the exposure then also takes place immediately, and is then fed as readout signal 29 to the image system 9.

The pulses represented in FIG. 6 and FIG. 10 and the pulses represented in FIG. 7 and FIG. 11 usually lie respectively on the same lines, as represented in the case of FIGS. 2 and 3. They have only been shown separately to give a better overview.

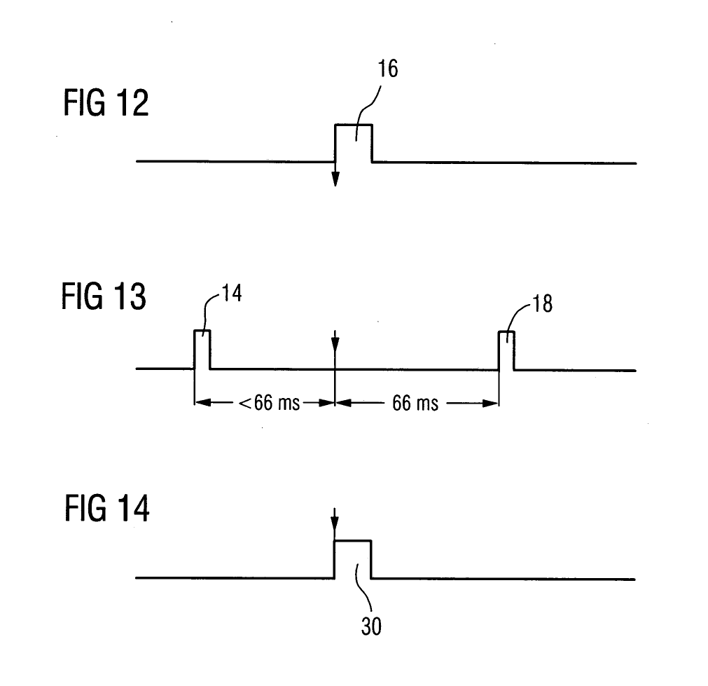

The activation according to an embodiment of the invention for another case is now explained in more detail on the basis of FIGS. 12 to 14. Shown in FIG. 12 is again the external trigger signal, in FIG. 13 the camera trigger and in FIG. 14 the control signal for the X-radiation. If the interval between a reset pulse 14 and the external trigger pulse 16 is then less than 66 ms (but more than 300 μs), an X-ray trigger pulse 30 which starts the X-radiation is generated directly by the trigger pulse 16. Subsequently, the transfer of the charge of the CCD image converter is triggered by the readout pulse 18.

If, however, as represented in FIGS. 6 to 11, the interval between the reset pulse 14 and the external trigger pulse 16 is more than 66 ms, the further reset pulse 27 is first set, bringing about a renewed dark readout 28. Only after completion of the reset pulse 27 is an X-ray trigger pulse then set, so that the X-radiation is started with something of a time offset with respect to the external trigger pulse 16. Then the transfer of the charge of the CCD image converter is triggered by the readout pulse 18.

A simpler construction of the system control 11 is obtained if, when the external trigger pulse 16 occurs, the X-ray trigger pulse 26 is delayed by the time which is required to transfer the charge from the light-sensitive region of the CCD image converter into the memory area. In the example described, this is 300 μs. As a result, this control does not need to consider whether the external trigger pulse 16 is removed from the reset pulse 14 by less or more than 66 ms. The readout pulse 18 also need not be set 66 ms after the trigger pulse 16. In the case of the example described on the basis of FIGS. 12 to 14, it can also be generated directly after completion of the X-radiation (not represented in these figures) and be triggered by it.

In the case of the known X-ray diagnostic systems, the reset pulses generated every 264 ms serve for the so-called dark triggering, to prevent an excessively high dark signal on the CCD image converter when no external triggers take place. If the external triggering takes place, the dark signal is dependent on the times of the triggering.

In the case of the X-ray diagnostic system according to an embodiment of the invention, on the other hand, a reset pulse 14 for dark triggering also takes place every 264 ms as long as there is no external triggering, but after that, triggered by the external trigger, a reset pulse 27 for dark triggering always takes place and the actual exposure takes place after that with a delay of about 300 μs. It is consequently ensured that all the images have the same dark signal (tending toward zero).

Only in the special situation in which the first external trigger pulse 16 comes within a window of 66 ms after the last periodic (264 ms) reset pulse 14 for the dark triggering would the camera be triggered twice. However, the camera needs 66 ms after a trigger to respond again meaningfully to the next trigger. This is to do with the readout mechanism of the CCD image converter. Otherwise, there are some regions in the CCD image converter that have already been read out and some that have not yet been read out. This would then lead to a different dark signal distribution in the actual image. Therefore, in this case the camera trigger (dark) is suppressed, in order that all the images of the series have exactly the same preconditions, that is to say dark signal contents.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An X-ray diagnostic system, comprising:
a CCD camera;
a device for generating external trigger pulses; and
a system control, formed in such a way that, in the absence of X-radiation, a readout of the CCD camera without a useful signal takes place at regular time intervals, wherein the system control is futher formed in such a way that, when an external trigger pulse occurs at a point in time at which no readout of the CCD camera is taking place, a read out without a useful signal is initially triggered and then an exposure of the CCD camera takes place, and wherein, when an external trigger pulse occurs at a point in time at which a readout of the CCD camera takes place, a readout without a useful signal is suppressed before an exposure of the CCD camera.

2. The X-ray diagnostic system as claimed in claim 1, wherein, when an external trigger pulse occurs at a point in time at which a readout of the CCD camera takes place, the X-ray diagnostic system is immediately triggered for the emission of X-radiation and the useful signal is subsequently read out.

3. The X-ray diagnostic system as claimed in claim 1, wherein, when an external trigger pulse occurs at a point in time at which no readout of the CCD camera takes place, a readout without a useful signal is initially carried out and then the X-ray diagnostic system is triggered for the emission of X-radiation.

4. The X-ray diagnostic system as claimed in claim 1, wherein the device for generating external trigger pulses is an ECG electrode.

5. The X-ray diagnostic system as claimed in claim 1, wherein the device for generating external trigger pulses is a phase-angle sensor.

6. The X-ray diagnostic system as claimed in claim 2, wherein, when an external trigger pulse occurs at a point in time at which no readout of the CCD camera takes place, a readout without a useful signal is initially carried out and then the X-ray diagnostic system is triggered for the emission of X-radiation.

7. The X-ray diagnostic system as claimed in claim 2, wherein the device for generating external trigger pulses is an ECG electrode.

8. The X-ray diagnostic system as claimed in claim 3, wherein the device for generating external trigger pulses is an ECG electrode.

9. The X-ray diagnostic system as claimed in claim 6, wherein the device for generating external trigger pulses is an ECG electrode.

10. The X-ray diagnostic system as claimed in claim 2, wherein the device for generating external trigger pulses is a phase-angle sensor.

11. The X-ray diagnostic system as claimed in claim 3, wherein the device for generating external trigger pulses is a phase-angle sensor.

12. The X-ray diagnostic system as claimed in claim 4, wherein the device for generating external trigger pulses is a phase-angle sensor.

13. The X-ray diagnostic system as claimed in claim 6, wherein the device for generating external trigger pulses is a phase-angle sensor.

14. The X-ray diagnostic system as claimed in claim 7, wherein the device for generating external trigger pulses is a phase-angle sensor.

15. The X-ray diagnostic system as claimed in claim 8, wherein the device for generating external trigger pulses is a phase-angle sensor.

16. The X-ray diagnostic system as claimed in claim 9, wherein the device for generating external trigger pulses is a phase-angle sensor.

17. An X-ray diagnostic system, comprising:
a CCD camera;
means for generating an external trigger pulse; and
means for, when an external trigger pulse is generated when no readout of the CCD camera is taking place, providing a readout without a useful signal before an exposure of the CCD camera, and for, when an external trigger pulse is generated when a readout of the CCD camera is taking place, supressing a readout without a useful signal before an exposure of the CCD camera.

18. The X-ray diagnostic system as claimed in claim 17, wherein the means for providing is formed in such a way that, in the absence of X-radiation, a readout of the CCD camera without a useful signal takes place at regular time intervals.

19. The X-ray diagnostic system as claimed in claim 17, wherein, when an external trigger pulse occurs at a point in time at which a readout of the CCD camera takes place, the X-ray diagnostic system is immediately triggered for the emission of X-radiation and the useful signal is subsequently read out.

20. The X-ray diagnostic system as claimed in claim 17, wherein, when an external trigger pulse occurs at a point in time at which no readout of the CCD camera takes place, a readout without a useful signal is initially carried out and then the X-ray diagnostic system is triggered for the emission of X-radiation.
